(19) United States
(12) Patent Application Publication
(10) Pub. No.: US 2009/0234282 A1
(43) Pub. Date: Sep. 17, 2009

(54) Outer Catheter Shaft to Balloon Joint

(75) Inventors: Eamonn McAndrew, Knocknacarra (IE); John Geraghty, Loughrea (IE); Michael Cummins, Roscam (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/049,687

(22) Filed: Mar. 17, 2008

(51) Int. Cl. A61M 25/10

(52) U.S. Cl. 604/103.06

(57) ABSTRACT

A balloon catheter having an improved profile by placing the proximal balloon neck inside the outer catheter shaft. This outer catheter shaft to balloon neck joint allows for a smaller outer diameter at the joint between the outer catheter shaft and the balloon, thus providing a reduced catheter profile with improved crossability, trackability and stiffness. The balloon may be a no-fold balloon having a uniform wall thickness, a uniform inner diameter, and a uniform outer diameter along its full length in an unexpanded configuration. A balloon with such uniform dimensions provides for a more flexible balloon by eliminating the thicker neck and taper portions of the balloon. Further, the no-fold aspect of the balloon reduces the profile of the balloon catheter thus resulting in improved crossability and trackability.

### FIELD OF THE INVENTION

The invention relates generally to a medical device. More particularly, the present invention relates to a catheter having an inflatable balloon at the distal end thereof joined on to the inside of an outer catheter shaft. In addition, the present invention relates to a balloon as bonded to the outer catheter shaft in an unexpanded configuration that has uniform dimensions including wall thickness, inner diameter, and outer diameter along its full length.

### BACKGROUND OF THE INVENTION

Cardiovascular disease, including atherosclerosis, is the leading cause of death in the U.S. One method for treating atherosclerosis and other forms of coronary narrowing is percutaneous transluminal coronary angioplasty, commonly referred to as “angioplasty” or “PTCA”. The objective in angioplasty is to enlarge the lumen of the affected coronary artery by radial hydraulic expansion. The procedure is accomplished by inflating a balloon of a balloon catheter within the narrowed lumen of the coronary artery. Radial expansion of the coronary artery occurs in several different dimensions, and is related to the nature of the plaque. Soft, fatty plaque deposits are flattened by the balloon, while hardened deposits are cracked and split to enlarge the lumen.

In addition to PTCA, balloon catheters are used for delivery of stents or grafts, therapeutic drugs (such as vaso-occlusion agents or tumor treatment drugs) and radiopaque agents for radiographic viewing. Other uses for such catheters are well known in the art.

In the design of catheter balloons, balloon characteristics such as strength, flexibility and compliance must be tailored to provide optimal performance for a particular application. In order to treat very tight stenoses with small openings, there has been a continuing effort to reduce the profile of the balloon catheter so that the balloon can reach and pass through the small opening of the stenoses.

Catheter balloons preferably have high flexibility and softness for improved ability to track the tortuous anatomy and cross lesions in the uninflated state. The anatomy of coronary arteries varies widely from patient to patient. Often a patient's coronary arteries are irregularly shaped, highly tortuous and very narrow. The tortuous configuration of the arteries may present difficulties to the physician in advancement of the balloon catheter to a treatment site. A highly tortuous coronary anatomy typically will present considerable resistance to advancement of the catheter over the guidewire. Therefore, it is important for the balloon catheter to be flexible and have a smooth profile to enable the balloon to be tracked to the treatment site. However, it is also important for a catheter shaft to be stiff enough to push the catheter into the vessel in a controlled manner from a position far away from the distalmost point of the catheter.

One factor that affects the profile of the balloon catheter is the joint between the proximal balloon neck and the outer catheter shaft. Typically, the balloon is welded or otherwise mechanically attached to the outer catheter shaft by placing the proximal balloon neck on the outside of the catheter shaft. By placing the balloon neck on the outside of the catheter shaft, the catheter presumably possesses a smoother profile for tracking the balloon to the treatment site since the “edge” created by the balloon to shaft joint is not pushed against the vessel wall while the balloon is being tracked through the patient's tortuous anatomy.

Another factor that affects the profile of the balloon catheter is the wall thickness of the balloon material. The profile of the deflated balloon is limited by the thickness of the neck and taper portions of the balloon. Usually, the neck and taper wall thicknesses of a torpedo-shaped angioplasty balloon are thicker than that of the body of the balloon due to the smaller diameter of the neck and taper portions. The thicker neck walls contribute to the overall thickness of the catheter, making tracking, crossing and re-crossing of lesions more difficult. Further, thick necks interfere with refolding of the balloon on deflation such that further inserting or withdrawing the deflated balloon may be difficult, occasionally even damaging the blood vessel. Reducing the wall thickness of the neck and taper portions reduces the overall profile of the deflated balloon.

Another factor which affects the profile of the balloon catheter is the balloon material itself. Angioplasty balloons are generally formed from relatively strong materials in order to withstand the pressures necessary for various procedures without failing. Typically, such characteristics require the use of a material that does not stretch appreciably. Use of polymeric materials that do not stretch appreciably consequently necessitates that the balloon is first formed by blow molding, and then the deflated balloon material, in the form of deflated wings, are folded around the catheter shaft prior to introduction of the balloon into the patient's body lumen. However, it may be desirable to employ balloons that do not have deflated folded wings, but which instead can be expanded to the working diameter within the patient's body lumen from an essentially wingless, cylindrical or tubular shape which conforms to the catheter shaft.

Thus, it is one aspect of the present invention to provide a balloon catheter with improved crossability and trackability, having a smooth reduced profile while simultaneously being sufficiently stiff to be tracked to the treatment site.

### BRIEF SUMMARY OF THE INVENTION

Embodiments of the present invention relate to a catheter having an outer catheter shaft, an inner catheter shaft, and a balloon. The outer catheter shaft has a proximal portion, a distal portion, an inside surface, an outside surface, and an inflation lumen extending there through. The inner catheter shaft has a proximal portion, a distal portion, an inside surface, an outside surface, and a guidewire lumen extending there through, wherein at least a portion of the inner catheter shaft is disposed within at least the distal portion of the outer catheter shaft. The balloon has a proximal end, a distal end, an inside surface, an outside surface, and an interior which is in fluid communication with the inflation lumen, wherein the outside surface of the balloon at the proximal end of the balloon is attached to the inside surface of the outer catheter shaft at the distal portion of the outer catheter shaft. In one embodiment of the present invention, the balloon has a uniform wall thickness, a uniform inner diameter, and a uniform outer diameter along its full length in an unexpanded configuration.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a side perspective view of a balloon delivery system with an embodiment of the present invention.

FIG. 2 is a cross-sectional view of the catheter of FIG. 1 taken along line A-A of FIG. 1.

FIG. 3 is a cross-sectional view of a catheter in accordance with another embodiment of the present invention taken along line A-A of FIG. 1.

FIG. 4 is an enlarged sectional view of the catheter of FIG. 1 taken along line B-B of FIG. 1.

FIG. 5 is a side perspective view of a balloon delivery system in accordance with another embodiment of the present invention.

FIG. 6 is an enlarged sectional view of the balloon delivery system of FIG. 5 along line C-C.

FIG. 7 is a cross-sectional view of the balloon of FIG. 5.

FIG. 8 is a side elevational view of tubing material for forming the balloon of FIG. 5.

### DETAILED DESCRIPTION OF THE INVENTION

Specific embodiments of the present invention are now described with reference to the figures, wherein like reference numbers indicate identical or functionally similar elements. The drawing in which an element first appears is typically indicated by the leftmost digit(s) in the corresponding reference number. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to limit the invention or the application and uses of the invention. It is noted that the invention is not limited to the specific embodiments described herein. Such embodiments are presented herein for illustrative purposes only. Additional embodiments will be apparent to persons skilled in the relevant art(s) based on the teachings contained herein. Although the description of the invention is in the context of treatment of blood vessels such as the coronary, carotid and renal arteries, the invention may also be used in any other body passageways where it is deemed useful. Furthermore, there is no intention to be bound by any expressed or implied theory presented in the preceding technical field, background, brief summary or the following detailed description.

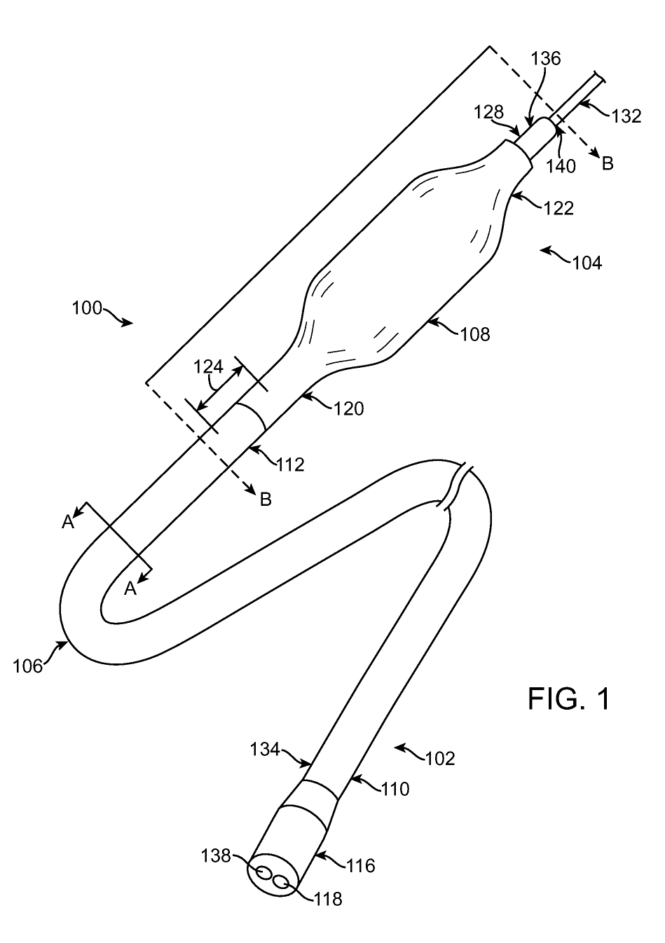

Embodiments of the present invention relate to a balloon catheter having an outer catheter shaft having a balloon mounted at the distal portion thereof. The profile of the balloon catheter is improved by placing the proximal balloon neck inside the outer catheter shaft. This outer shaft to balloon neck joint allows for a smaller outer diameter at the joint between the outer catheter shaft and the balloon, thus providing a reduced catheter profile in the joint transition area with improved crossability, trackability and stiffness. In one embodiment of the present invention, the balloon as bonded to the outer catheter shaft in an unexpanded configuration has uniform dimensions including wall thickness, inner diameter, and outer diameter along its full length. A balloon with such uniform dimensions provides for a more flexible balloon by eliminating the thicker neck and taper portions of the balloon. A balloon with such uniform dimensions is not folded prior to inflation, but is instead expanded to the working diameter from a generally cylindrical or tubular shape. This no-fold aspect of the balloon also reduces the profile of the balloon catheter, thus resulting in improved crossability and trackability. Another advantage associated with a balloon having uniform dimensions as described above is ease of manufacture. By eliminating the neck and taper regions from the balloon, the balloon may be formed from a simple mould design, which can be longer than the balloon length required, thus allowing multiple balloons be cut to length from balloons formed from the overlength mould. Further explanation and details will now be described with reference to FIGS. 1-8.

Referring now to FIGS. 1-4, an embodiment of a balloon catheter 100 according to the present invention is shown. Balloon catheter 100 includes a proximal portion 102, a distal portion 104, and an inflatable balloon 108 located at distal portion 104. Catheter 100 may be used for angioplasty procedures, stent delivery, and/or localized drug delivery.

Catheter 100 includes an outer catheter shaft 106 which includes at least one continuous lumen 214 extending from at or near its proximal end 110 to at or near its distal end 112 in order to provide for balloon inflation. Balloon 108 is located at or near distal end 112 of shaft 106, and a hub 116 is located at or near proximal end 110 of shaft 106. Hub 116 includes a balloon inflation port 118 to allow fluid communication between inflation lumen 214 and balloon 108 so that the balloon 108 may be inflated. Hub 116 will serve in a conventional manner to provide a luer or other fitting in order to connect the catheter 100 to a source of balloon inflation, such as conventional angioplasty activation device.

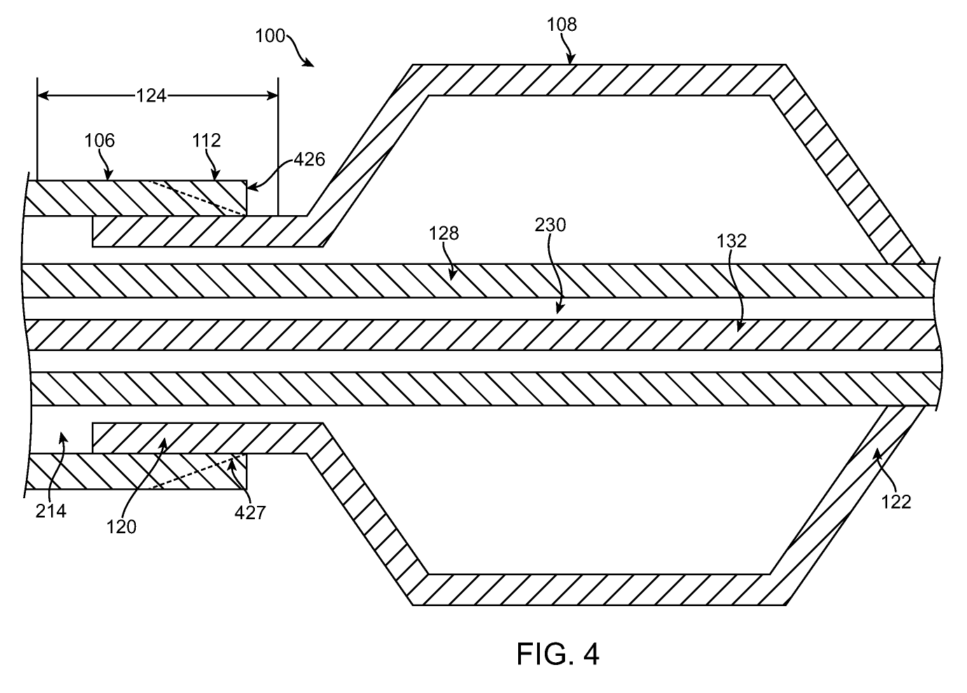

Balloon 108 includes a proximal neck end 120 and a distal neck end 122. At joint transition area 124, proximal neck end 120 of balloon 108 is placed inside and joined to the distal end 112 of outer catheter shaft 106, as shown in FIG. 4. Balloon 108 may be joined to outer catheter shaft 106 in any conventional manner, such as laser welding, adhesives, heat fusing, ultrasonic welding, or any other mechanical method. The profile of balloon catheter 100 is reduced by placing the proximal neck end 120 of balloon 108 inside outer catheter shaft 106 because such a configuration allows for a smaller outer diameter at joint transition area 124.

FIG. 4 is an enlarged sectional view at the location along line B-B of FIG. 1, and illustrates joint transition area 124 of catheter 100. As previously mentioned, typically an angioplasty balloon is welded or otherwise mechanically attached to the outer catheter shaft by placing the proximal balloon neck on the outside of the catheter shaft. By placing the proximal balloon neck on the outside of the catheter shaft, the catheter presumably possessed a smoother profile for tracking the balloon to the treatment site since the “edge” created by the balloon to shaft joint was not pushed against the vessel wall while the balloon was being tracked through the patient's tortuous anatomy. However, it is found that the edge 426 created by proximal neck end 120 of balloon 108 being placed inside the outer catheter shaft 106 did not hinder the crossability and trackability of catheter 100 while balloon 108 was being tracked through the patient's tortuous anatomy. Rather, having the proximal neck end 120 of balloon 108 placed inside the outer catheter shaft allows for a smaller outer diameter at joint transition area 124 and thus provides a reduced catheter profile with improved crossability, trackability and stiffness.

In addition, edge 426 may be modified in order to create a tapered edge 427. Tapered edge 427 is illustrated as a dotted line in FIG. 4. Tapered edge 427 creates a smoother joint transition area 124 to ensure that the distal edge of the catheter shaft is not pushed against the vessel wall while being tracked through the patient's tortuous anatomy. Edge 426 may also be rounded or otherwise modified such as by a necking or thinning operation to create a smoother joint transition area 124.

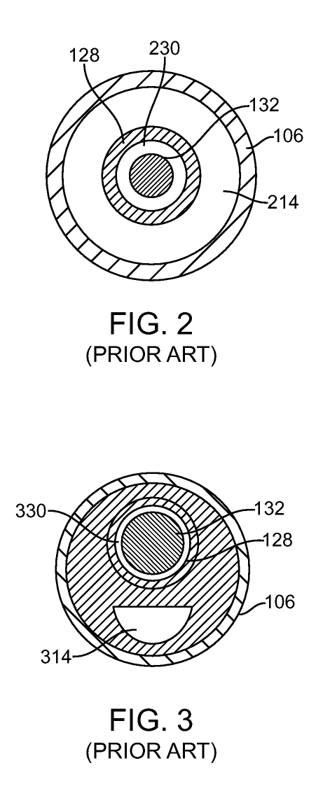

Referring now to FIG. 2, FIG. 2 is a cross-sectional view of a portion of balloon catheter 100 taken along line A-A of FIG. 1, and illustrates a coaxial dual lumen arrangement. In this embodiment, an inner or guidewire shaft 128 is disposed coaxially within outer catheter shaft 106. Inner shaft 128 includes at least one continuous lumen 230 extending from at or near its proximal end 134 to at or near its distal end 136 in order to track catheter 100 over a guidewire 132. As illustrated in FIG. 1, inner shaft 128 may extend the entire length of catheter 100, with a proximal guidewire port 138 provided in hub 116 and a distal guidewire port 140 provided at the distal portion 104 of catheter 100.

In the coaxial dual lumen arrangement illustrated in FIG. 2, inflation lumen 214 is created by a space between the outer surface of inner shaft 128 and the inner surface of outer catheter shaft 106. Lumen 214 is in fluid communication with an interior of balloon 108 such that balloon 108 may be inflated. FIG. 2 shows a guidewire 132 within guidewire lumen 230.

Other embodiments of balloon catheter 100 may have guidewire lumen 230 and inflation lumen 214 in other dual lumen arrangements, such as a circular guidewire lumen above a D-shaped inflation lumen or a circular guidewire lumen set above a crescent-shaped inflation lumen. For example, an alternative non-coaxial dual lumen arrangement is illustrated in FIG. 3. In this embodiment, inner shaft 128 may be disposed within outer catheter shaft 106 in a non-coaxial relationship. This alternate configuration results in a guidewire lumen 330 and an inflation lumen 314 being in a side-by-side arrangement through the length of the catheter. Guidewire 132 is shown within lumen 330 of inner shaft 128.

As previously described, the embodiments illustrated in FIGS. 1-3 include inner shaft 128 disposed within outer catheter shaft 106, with inner shaft 128 extending the entire length of catheter 100. Such a configuration is typically referred to as an over-the-wire (OTW) catheter. An OTW catheter's guidewire shaft runs the entire length of the catheter and is attached to, or enveloped within, an inflation shaft. Thus, the entire length of an OTW catheter is tracked over a guidewire during a PTCA procedure.

One skilled in the art can appreciate how the balloon to catheter joint of the present invention, described in detail above, may also be incorporated in a rapid exchange (RX) catheter. A RX catheter has a guidewire shaft that extends within only the distalmost portion of the catheter. Thus, during a PTCA procedure only the distalmost portion of a RX catheter is tracked over a guidewire.

Outer catheter shaft 106 may be formed of any appropriate polymeric material. In addition, inner shaft 128 may be made of any appropriate polymeric material. Non-exhaustive examples of material for outer catheter shaft 106 and inner shaft 128 include polyethylene, PEBAX, nylon or combinations of any of these, either blended or co-extruded. Preferred materials for shafts 106 and 128 are polyethylene, nylon, PEBAX, or co-extrusions of any of these materials.

Optionally, shafts 106 and 128 or some portion thereof may be formed as a composite having a reinforcement material incorporated within a polymeric body in order to enhance strength, flexibility, and/or toughness. Suitable reinforcement layers include braiding, wire mesh layers, embedded axial wires, embedded helical or circumferential wires, and the like. For example, at least a proximal portion of outer catheter shaft 106 may in some instances be formed from a reinforced polymeric tube. As a further alternative, at least a proximal portion of outer catheter shaft 106 may in some instances be formed from a metal, highly elastic, or super elastic hypotube material.

Balloon 108 can be any appropriate shape or size, and any material which is relatively elastic and deformable. Non-exhaustive examples for balloon 108 include polymers such as polyethylene, PEBAX, PET, nylon, polyurethane.

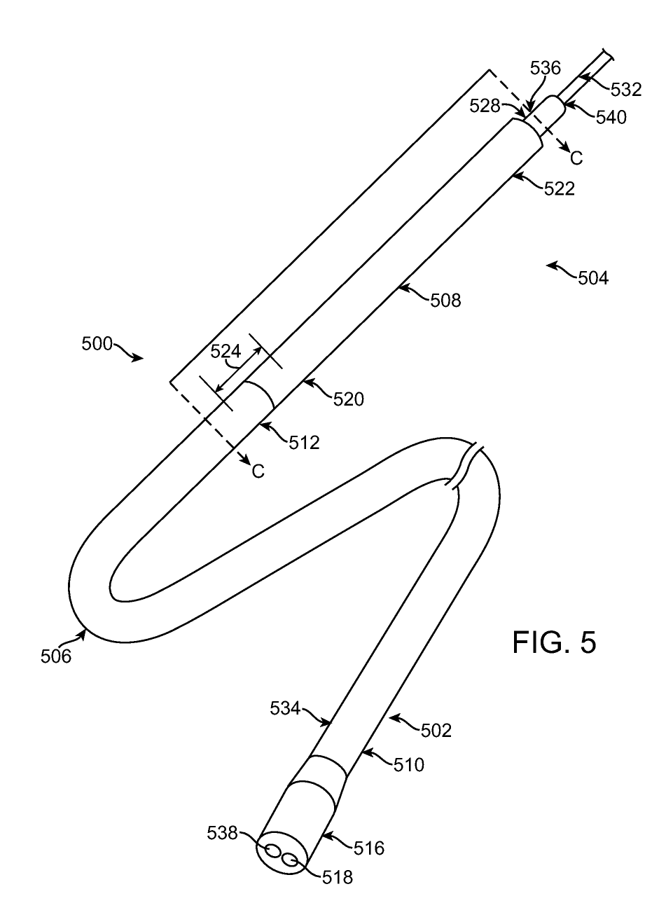

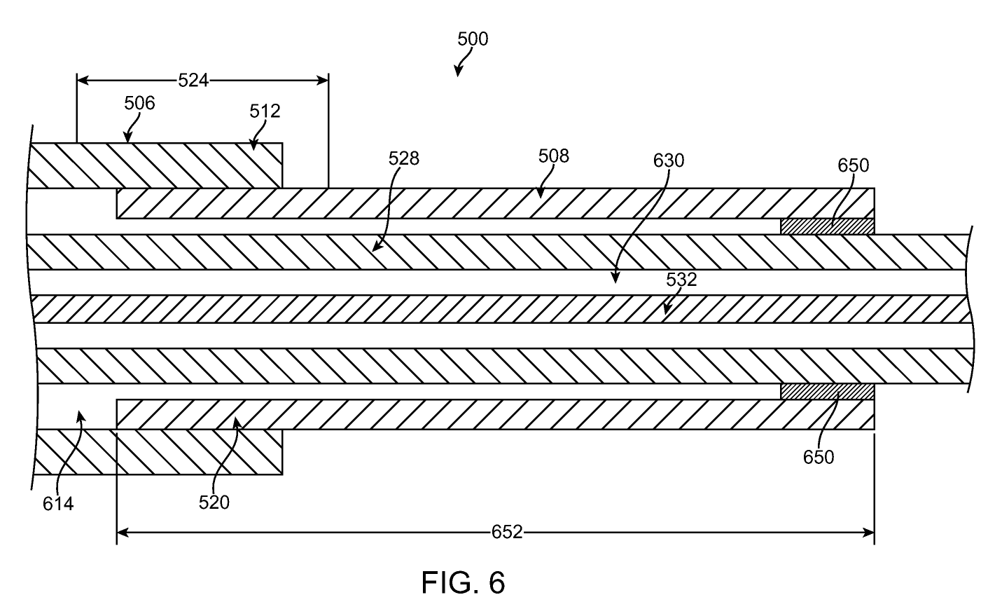

Now referring to FIGS. 5-7, another aspect of the present invention relates to a catheter 500 including a balloon 508 bonded to an outer catheter shaft 506. FIG. 5 illustrates balloon catheter 500 having a proximal portion 502 and a distal portion 504 with inflatable balloon 508 located at distal portion 504. As best shown in FIG. 6, balloon 508 has a length 652. In addition to forming the basis for balloon angioplasty procedures, catheter 500 may form the basis of a stent delivery system and/or a drug delivery system.

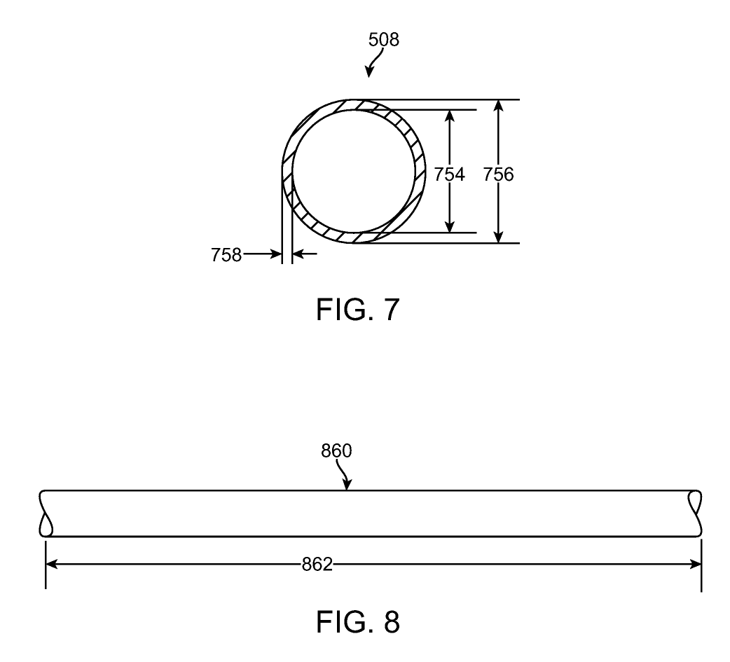

FIG. 7 is a cross-sectional view of the balloon of FIG. 5, and illustrates that balloon 508 has a wall thickness 758, an inner diameter 754, and an outer diameter 756. In an unexpanded configuration, wall thickness 758, inner diameter 754, and outer diameter 756 are uniform along the full length 652 of balloon 508. A balloon with such uniform dimensions provides for a more flexible balloon by eliminating the thicker neck and taper portions of the balloon. In addition, a balloon with such uniform dimensions is not folded prior to inflation, but is instead expanded to the working diameter from a generally cylindrical or tubular shape. This no-fold aspect of balloon 508 also reduces the profile of catheter 500, thus resulting in improved crossability and trackability.

Preferably, length 652 of balloon 508 is about 0.5 cm to about 4 cm and typically about 2 cm. When bonded to outer catheter shaft 506 in an unexpanded configuration, outer diameter 756 of balloon 508 is generally approximately 0.6 mm to about 0.9 mm along the full length 652 of balloon 508. Wall thickness 758 of balloon 508 is generally approximately 0.035 mm to about 0.05 mm, and inner diameter 754 is thus generally approximately 0.53 mm to about 0.8 mm. When inflated, balloon 508 assumes a torpedo shape having an inflated working outer diameter of about 1 mm to about 5 mm, typically about 3 mm, in order to enlarge the lumen of the affected coronary artery.

Catheter 500 includes outer catheter shaft 506 which includes at least one continuous lumen 614 extending from at or near its proximal end 510 to at or near its distal end 512 in order to provide for balloon inflation. Balloon 508 is located at or near distal end 512 of shaft 506, and a hub 516 is located at or near proximal end 510 of shaft 506. Hub 516 includes a balloon inflation port 518 to allow fluid communication between inflation lumen 614 and balloon 508 so that the balloon 508 may be inflated. Hub 516 will serve in a conventional manner to provide a luer or other fitting in order to connect the catheter 500 to a source of balloon inflation, such as conventional angioplasty activation device.

FIG. 6 is an enlarged sectional view at the location along line C-C of FIG. 5, and illustrates joint transition area 524 of catheter 500. Balloon 508 includes a proximal end 520 and a distal end 522. At joint transition area 524, proximal end 520 of balloon 508 is placed inside and joined to the distal end 512 of outer catheter shaft 506. Balloon 508 may be joined to outer catheter shaft 506 in any conventional manner, such as laser welding, adhesives, heat fusing, ultrasonic welding, or any other mechanical method. The profile of balloon catheter 500 is reduced by placing the proximal end 520 of balloon 508 inside outer catheter shaft 506 because such a configuration allows for a smaller outer diameter at joint transition area 524. Transition area 524 in FIG. 6 may also be rounded or otherwise modified such as by a necking or thinning operation to create a smoother transition joint.

Similar to the embodiment described above with respect to FIG. 2, catheter 500 includes an inner or guidewire shaft 528 disposed coaxially within outer catheter shaft 506. Inner shaft 528 includes at least one continuous lumen 630 extending from at or near its proximal end 534 to at or near its distal end 536 in order to provide a guidewire 532. As illustrated in FIG. 5, inner shaft 528 may extend the entire length of catheter 500, with a proximal guidewire port 538 provided in hub 516 and a distal guidewire port 540 provided at the distal portion of catheter 500. The distal end 522 of balloon 508 is joined to the inner shaft 528 at joint 650. Balloon 508 may be joined to inner shaft 528 in any conventional manner, such as laser welding, adhesives, heat fusing, ultrasonic welding, or any other mechanical method.

Inner shaft 528 and outer catheter shaft 506 may be arranged in various dual lumen configurations. Similar to the embodiment described above with respect to FIG. 2, inner shaft 528 and outer catheter shaft 506 may be arranged in a coaxial dual lumen configuration. In the coaxial dual lumen configuration, inflation lumen 614 is created by a space between the outer surface of inner shaft 528 and the inner surface of outer catheter shaft 506. Lumen 614 is in fluid communication with an interior of balloon 508 such that balloon 508 may be inflated.

Other embodiments of balloon catheter 500 may have guidewire lumen 630 and inflation lumen 614 in other dual lumen arrangements, such as a circular guidewire lumen above a D-shaped inflation lumen or a circular guidewire lumen set above a crescent-shaped inflation lumen. For example, similar to the configuration illustrated in FIG. 3, inner shaft 528 may be disposed within outer catheter shaft 506 in a non-coaxial relationship. This configuration results in a guidewire lumen 630 and an inflation lumen 614 being in a side-by-side arrangement through the length of the catheter.

As previously described, the embodiments illustrated in FIGS. 5-6 include inner shaft 528 disposed within outer catheter shaft 506, with inner shaft 528 extending the entire length of catheter 500. Such a configuration is typically referred to as an over-the-wire (OTW) catheter. An OTW catheter's guidewire shaft runs the entire length of the catheter and is attached to, or enveloped within, an inflation shaft. Thus, the entire length of an OTW catheter is tracked over a guidewire during a PTCA procedure.

One skilled in the art can appreciate how the balloon to catheter joint of the present invention, described in detail above, may also be incorporated in a rapid exchange (RX) catheter. A RX catheter has a guidewire shaft that extends within only the distalmost portion of the catheter. Thus, during a PTCA procedure only the distalmost portion of a RX catheter is tracked over a guidewire.

Outer catheter shaft 506 may be formed of any appropriate polymeric material. In addition, inner shaft 528 may be made of any appropriate polymeric material. Non-exhaustive examples of material for outer catheter shaft 506 and inner shaft 528 include polyethylene, PEBAX, nylon or combinations of any of these, either blended or co-extruded. Preferred materials for shafts 506 and 528 are polyethylene, nylon, PEBAX, or co-extrusions of any of these materials.

Optionally, shafts 506 and 528 or some portion thereof may be formed as a composite having a reinforcement material incorporated within a polymeric body in order to enhance strength, flexibility, and/or toughness. Suitable reinforcement layers include braiding, wire mesh layers, embedded axial wires, embedded helical or circumferential wires, and the like. For example, at least a proximal portion of outer catheter shaft 506 may in some instances be formed from a reinforced polymeric tube. As a further alternative, at least a proximal portion of outer catheter shaft 506 may in some instances be formed from a metal, highly elastic, or super elastic hypotube material.

As previously described, balloon 508 with such uniform dimensions as described above is not folded prior to inflation, but is instead expanded to the working diameter from a generally cylindrical or tubular shape. This no-fold aspect of balloon 508 reduces the profile of catheter 500 during insertion, thus resulting in improved crossability and trackability. Once balloon 508 is inflated, balloon 508 assumes a torpedo shape having a working outer diameter of approximately 3 mm in order to enlarge the lumen of the affected coronary artery. Upon deflation, shrinkage of the working outer diameter of the balloon occurs such that balloon 508 may be folded around catheter 500 and catheter 500 may be retracted from the patient.

The process of molding balloons for balloon catheters is generally known in the art. The process generally begins by placing an extruded tubular parison made of a drawable polymer having a specified diameter and wall thickness into the cavity of a mold. The balloon is then heated to a temperature in the range from the second-order transition temperature to the first-order transition temperature of the polymer used. Although the heating temperature will depend on the material, a temperature in the range of about 220-285° F. will generally suffice. While heated, the balloon is pressurized so that it will radially expand. A pressure in the range of about 300-450 psi is generally sufficient. The tubular product may also be axially elongated by stretching before, during, or after being radially expanded. Longitudinally stretching the material at a controlled velocity and distance thins out the material thickness in the balloon to the point where it will radially expand at the temperature and pressure in the balloon. The material of the balloon is thus biaxially oriented. A second process step is often used in balloon forming whereby the balloon inside the mould is exposed to a normalising or stress relieving step. This is usually achieved by applying a specific heat and pressure to the balloon for a fixed time period. Frequently the temperatures and pressures chosen for this process step are different to the heats and pressures used in the initial forming steps described above. This process step is often called a post-heat stage and its purpose is to impart uniformity to the balloons properties and to stabilise the balloon during storage and in further processing. Finally, the balloon is cooled in the mold to a temperature below the second-order transition temperature of the polymer. The completed balloon may then be removed from the mold.

In order to form balloon 508 having no-fold characteristics, the pressure is turned off during the post-heat stage (that is, after the balloon material is stretched both radially and longitudinally) in the balloon forming process to allow the balloon to shrink radially. In the post-heat stage of the manufacturing process, the balloon is in its stressed configuration. By turning off the pressure during this stage, the balloon in its stressed configuration is allowed to shrink radially to obtain an appropriate wingless unexpanded outer diameter.

By eliminating the importance of the neck and taper regions, a simpler mould design can be used to form balloon 508 since the taper and cone regions of the mould do not have to be tightly controlled as is the case with moulds for standard balloon designs. Balloon 508 is preferably formed from cut to length tubing. More specifically, FIG. 8 illustrates tubing 860 having a length 862. Tubing 860 Length 862 of tubing 800 is of such an amount that several balloons 508 may be formed from tubing 800. In other words, balloons 508 may be cut to length post-processing, thus allowing several balloons to be made in one manufacturing cycle.

In the alternative, balloon 508 may be formed by removing or cutting off the neck and taper regions of a torpedo-shaped balloon. In this method of manufacture, balloon 508 may be formed by heat-shrinking a blow-molded torpedo-shaped balloon to shrink the balloon to an appropriate wingless unexpanded outer diameter. Thereafter, the neck and taper regions may be cut off or otherwise removed to form balloon 508 having a uniform wall thickness, a uniform inner diameter, and a uniform outer diameter along its entire length.

Balloon 508 can be formed from any appropriate material which is relatively elastic and deformable. Non-exhaustive examples for balloon 108 include polymers such as polyethylene, PEBAX, PET, nylon, polyurethane, and polyamide. The preferred material for obtaining the no-fold aspect of balloon 508 is elastic, such as polyurethane.

Other advantages associated with balloon 508 having uniform dimensions as described above are ease of manufacture. The manufacturing process is simplified because balloon 508 eliminates the importance of the neck and taper regions during balloon forming.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A catheter comprising:
an outer catheter shaft having a proximal portion, a distal portion, an inside surface, an outside surface, and an inflation lumen extending there through;
an inner catheter shaft having a proximal portion, a distal portion, an inside surface, an outside surface, and a guidewire lumen extending there through, wherein at least a portion of the inner catheter shaft is disposed within at least the distal portion of the outer catheter shaft; and
a balloon having a proximal end, a distal end, an inside surface, an outside surface, and an interior which is in fluid communication with the inflation lumen, wherein the outside surface of the balloon at the proximal end of the balloon is attached to the inside surface of the outer catheter shaft at the distal portion of the outer catheter shaft.

2. The catheter according to claim 1, wherein the inner shaft extends the entire length of the catheter and the proximal portion of the inner catheter is disposed within the proximal portion of the outer catheter shaft.

3. The catheter according to claim 1, wherein the balloon has a uniform wall thickness, a uniform inner diameter, and a uniform outer diameter along a full length of the balloon in an unexpanded configuration.

4. The catheter according to claim 3, wherein the outer diameter is small enough to be tracked to a treatment site within a patient's vasculature without folding or wrapping the balloon around the catheter.

5. The catheter according to claim 4, wherein the outer diameter is approximately 0.6 mm.

6. The catheter according to claim 5, wherein the balloon assumes a torpedo shape having a working outer diameter of approximately 3 mm when inflated.

7. The catheter according to claim 3, wherein the balloon is constructed from polyurethane.

8. The catheter according to claim 1, wherein the inner catheter shaft is disposed coaxially within the outer catheter shaft such that the inflation lumen is formed between the inner catheter shaft and the outer catheter shaft.

9. The catheter according to claim 1, wherein the inner catheter shaft is disposed non-coaxially within the outer catheter shaft such that the inflation lumen and the guidewire lumen are in a side-by-side arrangement through the length of the catheter.

10. The catheter according to claim 1, wherein an edge is formed at the distal portion of the outer catheter shaft, the edge being tapered to provide a smooth joint transition area.

11. A catheter comprising:
an outer catheter shaft having a proximal portion, a distal portion, an inside surface, an outside surface, and an inflation lumen extending there through;
an inner catheter shaft having a proximal portion, a distal portion, an inside surface, an outside surface, and a guidewire lumen extending there through; and
a balloon having a proximal end, a distal end, an inside surface, an outside surface, and an interior which is in fluid communication with the inflation lumen, the balloon having a uniform wall thickness, a uniform inner diameter, and a uniform outer diameter along its full length in an unexpanded configuration, wherein the outside surface of the balloon at the proximal end of the balloon is attached to the inside surface of the outer catheter shaft at the distal portion of the outer catheter shaft.

12. The catheter according to claim 11, wherein the outer diameter is small enough to be tracked to a treatment site within a patient's vasculature without folding or wrapping the balloon around the catheter.

13. The catheter according to claim 12, wherein the outer diameter is approximately 0.6 mm.

14. The catheter according to claim 12, wherein the balloon assumes a torpedo shape having a working outer diameter of approximately 3 mm when inflated.

15. The catheter according to claim 11, wherein the balloon is constructed from polyurethane.

16. The catheter according to claim 11, wherein an edge is formed at the distal portion of the outer catheter shaft, the edge being tapered to provide a smooth joint transition area.

17. A method for forming a catheter balloon, the method including the steps of:
placing a tubular balloon into the cavity of a mold;
heating the balloon from a base temperature to an elevated temperature, wherein the
elevated temperature is greater than a second order transition temperature for the balloon;
pressurising the balloon while the balloon is at the elevated temperature such that it radially expands;
longitudinally stretching the balloon while the balloon is at the elevated temperature;
causing the balloon to shrink radially to a wingless unexpanded outside diameter by
turning off the pressure while the balloon is held at the elevated temperature,
cooling the balloon while in the mold to a temperature at or below the base temperature; and
removing the balloon from the mold.

18. The method of claim 17, wherein the wingless unexpanded outer diameter of the balloon is small enough to be tracked to a treatment site within a patient's vasculature without folding or wrapping the balloon around the catheter.

19. The method of claim 17, wherein the wingless unexpanded outer diameter of the balloon is approximately 0.6 mm.

20. The method of claim 17, wherein the balloon is constructed from polyurethane.
